(19) United States
(12) Patent Application Publication
(10) Pub. No.: US 2007/0053877 A1
(43) Pub. Date: Mar. 8, 2007

(54) Methods of treating idiopathic pulmonary fibrosis

(75) Inventors: Michael Crager, Menlo Park, CA (US); Scott Harkonen, San Francisco, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: INTERMUNE, INC., Brisbane, CA (US)

(21) Appl. No.: 11/487,733

(22) Filed: Jul. 17, 2006

Related U.S. Application Data
(63) Continuation of application No. 10/525,583.

(51) Int. Cl. A61K 38/21

(52) U.S. Cl. 424/85.6

(57) ABSTRACT

The present application provides methods of treating idiopathic pulmonary fibrosis (IPF); methods of increasing survival time in an individual with IPF, and methods of reducing risk of death in an individual with IPF. The methods generally involve administering a therapeutically effective amount of IFN-γ to an individual with IPF.

### FIELD OF THE INVENTION

This invention is in the field of therapy of treating idiopathic pulmonary fibrosis.

### BACKGROUND OF THE INVENTION

Pulmonary fibrosis can be caused by a number of different conditions, including sarcoidosis, hypersensitivity pneumonitis, collagen vascular disease, and inhalant exposure. The diagnosis of these conditions can usually be made by careful history, physical examination, chest radiography, including a high resolution computer tomographic scan (HRCT), and open lung or transbronchial biopsies. However, in a significant number of patients, no underlying cause for the pulmonary fibrosis can be found. These conditions of unknown etiology have been termed idiopathic interstitial pneumonias. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

The logic of dividing idiopathic interstitial pneumonias into these categories is based not only on histology, but also on the different response to therapy and prognosis for these different entities. DIP is associated with smoking and the prognosis is good, with more than 70% of these patients responding to treatment with corticosteroids. NSIP patients are also frequently responsive to steroids and prognosis is good, with 50% of patients surviving to 15 years. In contrast, the UIP histologic pattern is associated with a poor response to therapy and a poor prognosis, with survival of only 3-5 years.

Idiopathic pulmonary fibrosis (IPF) is the most common form of idiopathic interstitial pneumonia and is characterized by the UIP pattern on histology. IPF has an insidious onset, but once symptoms appear, there is a relentless deterioration of pulmonary function and death within 3-5 years after diagnosis. The mean age of onset is 6065 and males are affected approximately twice as often as females. Prevalence estimates are 13.2-20.2 per 100,000. The annual incidence is estimated to be 7.4-10.7 per 100,000 new cases per year.

Published evidence suggests that less than 20% of patients with IPF respond to steroids. In patients who have failed treatment with steroids, cytotoxic drugs such as azathioprine or cyclophosphamide are sometimes added to the steroid treatment However, a large number of studies have shown little or no benefit of these drugs. There are currently no drugs approved for treatment of IPF.

There is a need in the art for methods of treating IPF. The present invention addresses this need.

Literature

WO 01/34180; Ziesche et al. (1999) N. Engl. J. Med. 341:1264-1269; du Bois (1999) N. Engl. J. Med. 341:1302-1304; U.S. Pat. No. 6,294,350; EP 795,332; King (2000) N. Engl. J. Med. 342:974-975; Ziesche and Block (2000) Wien. Klin Wochenschr. 112:785-790; Stern et al. (2001) Chest 120:213-219; Gay et al. (1998) Am. J. Respir. Crit. Care Med. 157:1063-1072; Dayton et al. (1993) Chest 103:69-73.

### SUMMARY OF THE INVENTION

The present invention provides methods of treating idiopathic pulmonary fibrosis (IPF); methods of increasing survival time in an individual with IPF; and methods of reducing risk of death in an individual with IPF. The methods generally involve administering a therapeutically effective amount of IFN-γ to an individual with IPF.

### BRIEF DESCRIPTION OF THE DRAWINGS

FIG. 1 depicts the survival probability in patients, having less than 55% of predicted forced vital capacity, treated with IFN-γ 1b or placebo.

FIG. 2 depicts the survival probability in patients, having at least 55% predicted forced vital capacity, treated with IFN-γ 1b or placebo.

### DEFINITIONS

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual,” “host,” “subject,” and “patient,” used interchangeably herein, refer to a mammal, particularly a human

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a method” includes a plurality of such methods and reference to “an IFN-γ dose” includes reference to one or more doses and equivalents thereof known to those skilled in the art, and so forth

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating idiopathic pulmonary fibrosis (IPF); methods of increasing survival time in an individual with IPF; and methods of reducing risk of death in an individual with IPF. The methods generally involve administering a therapeutically effective amount of IFN-γ to an individual with IPF.

Methods of Treating Idiopathic Pulmonary Fibrosis

The present invention provides methods of treating idiopathic pulmonary fibrosis (IPF). The methods generally involve administering an effective amount of IFN-γ to an individual having IPF.

In some embodiments, a diagnosis of IPF is confirmed by the finding of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101.

In other embodiments, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy (or, if present, is not extensive enough to be visible on chest x-ray). A diagnosis of definite IPF is made if characteristics (1), (2), and (3) are met. A diagnosis of probable IPF is made if characteristics (1) and (3) are met

IFN-γ is administered in an effective amount. In some embodiments, an effective amount of IFN-γ is an amount effective to increase the probability of survival of an individual having IPF by at least about 10%, at least about 15%, at least about 20%, or at least about 25%, or more, compared to the expected probability of survival without administration of IFN-γ. Thus, the increased probability of survival of an individual having IPF and administered with an effective amount of IFN-γ is at least about 10%, at least about 15%, at least about 20%, or at least about 25%, or more, compared to the expected probability of survival without administration of IFN-γ.

In some embodiments, an effective amount of IFN-γ is an amount that reduces the risk of death in an individual with IPF. The risk of death in an individual having IPF and treated with IFN-γ is reduced at least 2-fold, at least 2.5-fold, at least 3-fold, at least 3.5-fold, or at least 4-fold, or less, compared to the expected risk of death in an individual having IPF and not treated with IFN-γ.

Interferon-Gamma

The nucleic acid sequences encoding IFN-γ polypeptides may be accessed from public databases, e.g. Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank XI 3274); and Rinderknecht et al. (1984) J. Biol. Chem. 259:6790.

IFN-γ1b (Actimmune®; human interferon) is a single-chain polypeptide of 140 amino acids. It is made recombinantly in E. coli and is unglycosylated. Rinderknecht et al. (1984) J. Biol. Chem. 259:6790-6797.

The IFN-γ to be used in the compositions of the present invention may be any of natural IFN-γs, recombinant IFN-γs and the derivatives thereof so far as they have a IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra. consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG which, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present Extraneous sequences can be substituted for the amino acid sequence following amino acid 155 without loss of activity. See, for example, U.S. Pat. No. 5,690,925, herein incorporated by reference. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. In one embodiment, the invention contemplates the use of IFN-γ variants with one or more non-naturally occurring glycosylation and/or pegylation sites that are engineered to provide glycosyl- and/or PEG-derivatized polypeptides with reduced serum clearance, such as the IFN-γ polypeptide variants described in International Patent Publication No. WO 01/36001. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Dosages, Formulations, and Routes of Administration

IFN-γ is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In the subject methods, the active agent(s) may be administered to the host using any convenient means capable of resulting in the desired therapeutic effect Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Effective dosages of IFN-γ can range from about 0.5 μg/m² to about 500 μg/m², usually from about 1.5 μg/m² to 200 μg/m², depending on the size of the patient. This activity is based on 10⁶ international units (FU) per 50 μg of protein.

Those of skill will readily appreciate that dose levels can vary as a function of the specific compound, the severity of the symptoms and the susceptibility of the subject to side effects. Preferred dosages for a given compound are readily determinable by those of skill in the art by a variety of means. A preferred means is to measure the physiological potency of a given compound.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, dosage groups (based on administration of 200 μg IFN-γ per dose) range from about 150 μg/m² to about 80 μg/m². For example, dosage groups range from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m².

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide (e.g., a polynucleotide encoding IFN-γ), it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells. Of particular interest in these embodiments is use of a liver-specific promoter to drive transcription of an operably linked IFN-γ coding sequence preferentially in liver cells.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

In particular embodiments of interest, IFN-γ is administered as a solution suitable for subcutaneous injection. For example, IFN-γ is in a formulation containing 40 mg mannitol/mL, 0.72 mg sodium succinate/mL, 0.10 mg polysorbate 20/mL. In particular embodiments of interest, IFN-γ is administered in single-dose forms of 200 μg/dose subcutaneously.

Multiple doses of IFN-γ can be administered, e.g., IFN-γ can be administered once per month, twice per month, three times per month, once per week, twice per week, three times per week, four times per week, five times per week, six times per week, or daily, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In particular embodiments of interest, IFN-γ is administered three times per week over a period of at least about 1 year.

Additional Agents

In some embodiments, IFN-γ is co-administered with one or more additional agents. Suitable additional agents include corticosteroids, such as prednisone. When co-administered with IFN-γ, predisone is administered in an amount of 7.5 mg or 15 mg daily, administered orally.

Subjects Suitable for Treatment

The subject methods are suitable for treatment of individuals diagnosed as having IPF. The methods are also suitable for treatment of individuals having IPF who were previously treated with corticosteroids within the previous 24 months, and who failed to respond to previous treatment with corticosteroids. Subjects that are particularly amenable to treatment with a method are those that have at least 55% of the predicted FVC. Also suitable for treatment are subject that have at least 60% of the predicted FVC, or from 55% to 70% of the predicted FVC. The percent predicted FVC values are based on normal values, which are known in the art See, e.g., Crapo et al. (1981) Am. Rev. Respir. Dis. 123:659-664. FVC is measured using standard methods of spirometry.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Treatment of IPF

Materials and Methods

Study Population

Male and female patients were those ages 20-79 with idiopathic pulmonary fibrosis. Patients aged 20-34 were diagnosed by open or video-assisted thoracoscopic (VATS) lung biopsy or by transbronchial biopsy to be eligible. Diagnosis was made by high resolution computer tomographic scan showing definite or probable IPF and either open or VATS lung biopsy showing definite or probable usual interstitial pneumonia (UIP) within 30 months prior to screening; or non-diagnostic transbronchial biopsy to exclude other conditions within 30 months prior to screening and abnormal PFTs (reduced FVC or decreased DL_{co} or impaired gas exchange with rest or exercise) and 2 of the following: age greater than 50 years, insidious onset of otherwise unexplained dyspnea on exertion, and bibasilar, inspiratory crackles (dry or “Velcro” type in quality). Patients had clinical symptoms consistent with IPF of ≧3 months duration and had worsening disease within the past year.

Patients included in the study failed to show improvement after an adequate course of steroids that was completed within the 24 months prior to treatment in the present protocol. Failure to show “improvement” refers to failure to show an increase of ≧10% in the percent predicted FVC from baseline value (before steroids started) to any point after the steroid administration period and before randomization. Patients who showed ≧10% improvement, then returned to the baseline value, despite the continuation of the same dose of steroids that was associated with improvement, were eligible. For patients with a diagnosis of IPF established within the past year, an “adequate course” of steroids is a total oral dose of 1800 mg of prednisone or its equivalent administered over a period of no less than 1 month and no greater than 3 months. For patients with a diagnosis of IPF established more than 1 year prior to treatment, an “adequate course” of steroids is a total oral dose of 1800 mg of prednisone or its equivalent administered within a 6 month period.

Exclusion Criteria

Patients with any of the following were excluded from the study: (1) History of clinically significant environmental exposure known to cause pulmonary fibrosis (drugs, asbestos, beryllium, radiation, domestic birds, etc.); (2) Known explanation for interstitial lung disease, other than IPF, including but not limited to radiation, sarcoidosis, hypersensitivity pneumonitis, bronchiolitis obliterans organizing pneumonia (BOOP), and cancer; (3) Diagnosis of any connective tissue disease (scleroderma, systemic lupus erythematosus, rheumatoid arthritis, etc.) according to American College of Rheumatology criteria; (4) Forced expiratory volume in the first second (FEV₁)/forced vital capacity (FVC) ratio <0.6 at Screening (post-bronchodilator); (5) Patients with a residual volume >120% of predicted at Screening (pre-bronchodilator); (6) Evidence of active infection, including bronchitis, sinusitis, urinary tract infection (UTI), and cellulitis within 1 week prior to treatment; (7) Any condition other than IPF which, in the opinion of the site Principal Investigator (PI), is likely to result in the death of the patient within the next year, (8) History of unstable or deteriorating cardiac or neurologic disease, including but not limited to: a) Myocardial infarction, coronary artery bypass surgery, or angioplasty within the past 6 months; b) Congestive heart failure requiring hospitalization within the past 6 months; c) Uncontrolled arrhythmias; d) Transient ischemic attacks (TIAs); (9) Any cardiac or neurologic condition which, in the opinion of the site PI, might be significantly exacerbated by the known flu-like syndrome associated with the administration of IFN-γ 1b; (10) History of peripheral vascular disease which, in the opinion of the site PI, might be exacerbated by the known flu-like syndrome associated with the administration of IFN-γ 1b; (11) History of CNS disorder which, in the opinion of the site PI, might be exacerbated by the known flu-like syndrome associated with the administration of IFN-γ 1b. In addition, patients with the following conditions should be excluded: a) History of multiple sclerosis; b) Seizures within the past 10 years or taking anti-seizure medication; (12) History of severe or poorly controlled diabetes; (13) Pregnancy or lactation. Females of childbearing potential were required to have a negative serum or urine pregnancy test prior to treatment and must agree to practice abstinence or prevent pregnancy by at least a barrier method of birth control for the duration of the study; (14) Any of the following liver function test criteria above specified limits: Total bilirubin ≧1.5×ULN; aspartate or alanine aminotransferases (AST, SGOT or ALT, SGPT) >3×ULN; alkaline phosphatase >3×ULN; and albumin <3.0 mg/dL at Screening; (15) Hematology outside of specified limits: WBC <2,500/mm³, hematocrit <30% or >59%, platelets <100,000/mm³ at Screening; (16) Creatinine >1.5×ULN at Screening; (17) Prior treatment with IFN-γ 1b, beta interferon (Avonex), or other interferons; (18) Investigational therapy for any indication within 28 days prior to treatment; (19) Use of azathioprine, colchicine, cyclophosphamide, cyclosporine, D-penicillamine, methotrexate, or N-acetyl cysteine within 6 weeks prior to treatment; (20) Investigational therapy, including pirfenidone, within 6 months prior to treatment; (21) Patients who, in the opinion of the site PI, are not suitable candidates for enrollment or would not comply with the requirements of the study.

Primary Endpoints

The primary endpoints were progression-free survival time (e.g., time from baseline to death or disease progression). Disease progression was defined as the occurrence of either of the following: a decrease in % predicted FVC of 10% or more compared to baseline on two consecutive occasions 4-14 weeks apart; an increase in A-a gradient of 5 mm Hg or more compared to baseline on two separate occasions 4-14 weeks apart.

Secondary Endpoints

Secondary endpoints were as follows: (1) Transitional dyspnea index (TDI) at Week 48; (2) Progression-free survival time with disease progression defined by the presence of any two of the following: (a) Decrease of 10% or more in percent predicted FVC; (b) Increase of 5 mmHg or more in A-a gradient; (c) Decrease of 15% or more in single breath DL_{CO}; (3) Change from baseline to Week 48 in DL_{CO} (numerical value); (4) Change from baseline to Week 48 in FVC (numerical value); (5) Change from baseline to Week 48 in A-a gradient (numerical value); (6) Quality of Life as assessed by the St. George's Respiratory Questionnaire total score change from baseline to Week 48; (7) Survival time from randomization through clinical data cutoff, summarized by treatment group; (8) Response status of lung fibrosis as assessed by HRCT (better, same, worse) at 48 weeks compared to baseline; (9) Most severe requirement for use of outpatient oxygen (none, with activity, at rest) during each month on study, compared between treatment groups.

Safety Observations

Patients were evaluated at Weeks 1 and 2, at the monthly visits thereafter to assess adverse events. Laboratory tests, including a complete blood count; routine chemistry tests including creatinine; liver function tests; cholesterol; triglycerides; and urinalysis were measured at baseline, Weeks 1, 2, 4, 12, and every 12 weeks thereafter. Thyroid function tests were performed at baseline, Week 12 (Month 3), Week 24 (Month 6), Week 48 and every 6 months thereafter. Any Serious Adverse Events and Grade IV toxicities were reported in real time to the Sponsor or its designee regardless of relationship to study drug.

Efficacy Observations

Patients were subjected to pulmonary function tests (spirometry, DL_{CO}) and resting arterial blood gases assessed at baseline and every 12 weeks (3 months) thereafter. Dyspnea (modified MRC scale) was assessed at baseline and every 4 weeks (monthly) thereafter. Dyspnea (BDI/TDI and the UCSD Shortness of Breath Questionnaire) was assessed at baseline and every 12 weeks (3 months) thereafter. Quality-of-life questionnaires (SF-36 and SGRQ) were given at baseline and every 12 weeks (3 months) thereafter. Oxygen use was monitored daily. HRCT scans were done at baseline and at 48 weeks.

Study Design

A randomized, double-blind, placebo-controlled study of 330 patients with randomization balanced by study site and for smoking status. Patients were assigned to one of two groups: Group 1: 200 μg IFN-γ 1b subcutaneous administration three times a week; Group 2: placebo, subcutaneous administration of saline three times a week (tiw).

The study comprised three periods: the Screening Period (up to 28 days duration), the Study Period (up to 37 months duration), and the Long-Term Follow-Up Period (5 years). During the Study Period, patients were dosed with study drug tiw for up to 3 years. The final analysis was conducted when the 306^{th} patient had been followed for 48 weeks and included data from all patients randomized Patients who withdrew from study treatment early had a complete post-treatment evaluation visit 12 weeks (3 months) after their last treatment and then visited every 12 weeks (3 months) thereafter for assessment of primary and secondary endpoints as well as medications used to treat IPF.

Study treatment continued until the Study Completion Visit, and the Study Period ended with the Follow-Up Visit conducted 28 days following the Study Completion Visit Subsequent to the Study Period, patient vital status will be assessed every 6 months for 5 years during the Long-Term Follow-Up Period. A Data and Safety Monitoring Board (DSMB) monitored patient safety regularly.

Patients may be taking up to 15 mg of prednisone per day at study entry and should remain on the same dose (entry level) of steroids throughout the study. Treatment with colchicine, cytotoxic drugs, cyclosporine, N-acetyl cysteine, or other experimental therapies will not be allowed.

Data Analysis

The primary efficacy endpoint is the time to first occurrence of disease progression or death, as assessed by the Cox proportional hazards model.

Results

No statistically significant difference was apparent in the progression-free survival times of the treatment and placebo groups. Nevertheless, a statistically significant improvement in probability of survival was apparent in certain subpopulations of the treatment and placebo groups.

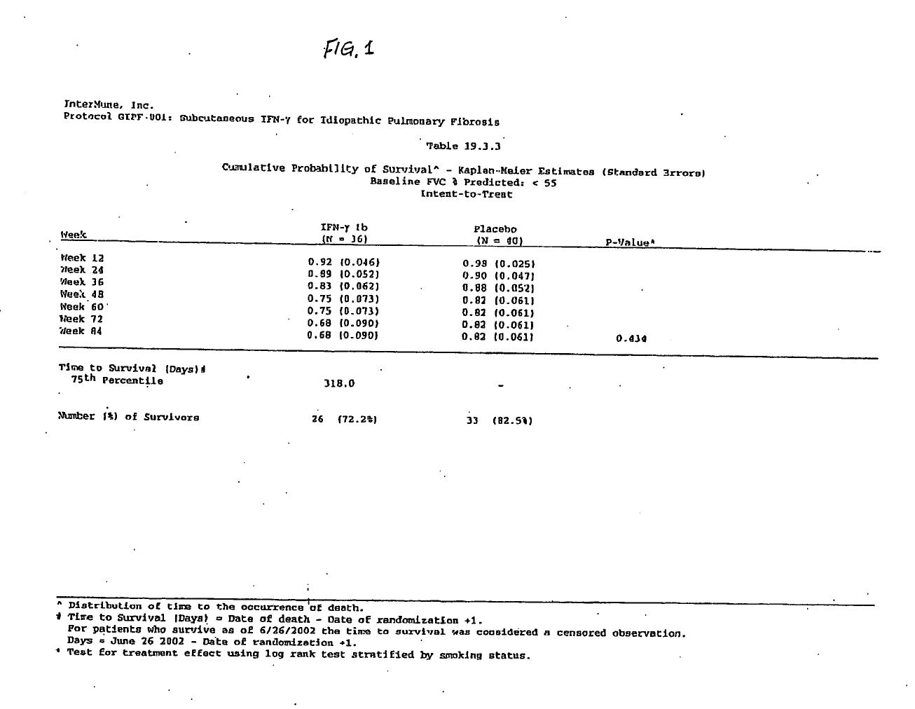

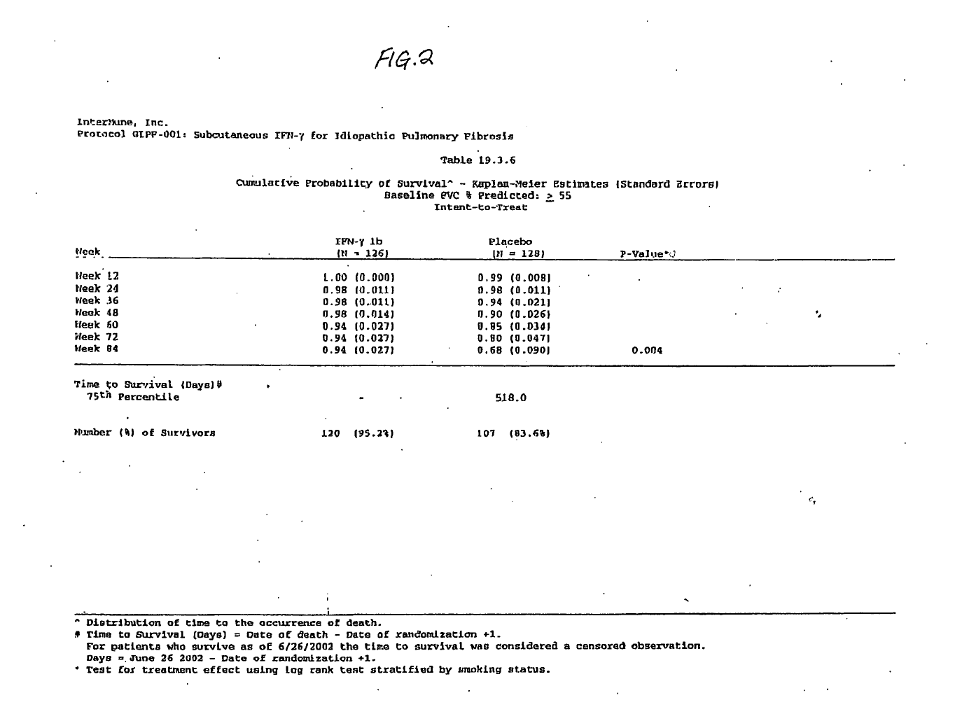

The results for patient survival are shown in FIGS. 1 and 2. FIG. 1 presents the data for individuals who had a % predicted FVC of less than 55 at the beginning of treatment Individuals (N=36) treated with IFN-γ 1b and having a % predicted FVC of less than 55% had a probability of 72.2% survival, while placebo controls (N=40), had an 82.5% probability of survival (p=0.434). Thus, the observed risk of death among individuals with IPF and having an FVC of less than 55% of the predicted normal value was 27.8%, while the risk of death of the placebo controls was 17.5%. There is no statistical evidence that IFN-γ 1b has a survival effect in these patients.

FIG. 2 presents the date for individuals who had a % predicted FVC of 55 or greater at the beginning of treatment. Individuals (N=126) treated with IFN-γ 1b and having a % predicted FVC of 55 or greater had a probability of 95.2% survival, while placebo controls (N=128) had a probability of 83.6% survival (p=0.004). Thus, the risk of death among individuals with IPF and having an FVC of 55% or greater of the predicted normal value was 4.8%, while the risk of death of the placebo controls was 16.4%. Thus, in this group, the observed risk of death was decreased by more than 3 fold. There is strong statistical evidence that IFN-γ 1b has a positive survival effect in these patients.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating idiopathic pulmonary fibrosis (IPF) in an individual, the method comprising administering to the individual an effective amount of IFN-γ
wherein the individual has a forced vital capacity (FVC) that is at least about 55% of the normal predicted value.

2. The method of claim 1, wherein the method further comprises administering a corticosteroid to the individual.

3. The method of claim 1, wherein the probability of survival of the individual is at least about 10% greater than an expected probability of survival without administration of IFN-γ.

4. The method of claim 1, wherein the probability of survival of the individual is at least about 15% greater than an expected probability of survival without administration of IFN-γ.

5. The method of claim 1, wherein the risk of death of the individual is at least two-fold less than an expected risk of death without administration of IFN-γ.

6. The method of claim 1, wherein the risk of death of the individual is at least four-fold less than an expected risk of death without administration of IFN-γ.

7. The method of claim 1, wherein IFN-γ is administered in a dose of about 80 μg/m² to about 90 μg/m².

8. The method of claim 1, wherein IFN-γ is administered in a dose of about 200 μg.

9. The method of claim 7 or 8, wherein IFN-γ is administered three times weekly.

10. The method of claim 9, wherein IFN-γ is administered by subcutaneous administration.

11. A method for increasing probability of survival of an individual having idiopathic pulmonary fibrosis (IPF), the method comprising administering to the individual an effective amount of IFN-γ
wherein the individual has a forced vital capacity (FVC) that is at least about 55% of the normal predicted value.

12. The method of claim 11, wherein the method further comprises administering a corticosteroid to the individual.

13. The method of claim 11, wherein the probability of survival of the individual is at least about 10% greater than an expected probability of survival without administration of IFN-γ.

14. The method of claim 11, wherein the probability of survival of the individual is at least about 15% greater than an expected probability of survival without administration of IFN-γ.

15. The method of claim 11, wherein IFN-γ is administered in a dose of about 80 μg/m² to about 90 μg/m².

16. The method of claim 11, wherein IFN-γ is administered in a dose of about 200 μg.

17. The method of claim 15 or 16, wherein IFN-γ is administered three times weekly.

18. The method of claim 17, wherein IFN-γ is administered by subcutaneous administration.

19. A method of reducing the risk of death of an individual having idiopathic pulmonary fibrosis (IPF) in an individual, the method comprising administering to the individual an effective amount of IFN-γ
wherein the individual has a forced vital capacity (FVC) that is at least about 55% of the normal predicted value.

20. The method of claim 19, wherein the method further comprises administering a corticosteroid to the individual.

21. The method of claim 19, wherein the risk of death of the individual is at least two-fold less than an expected risk of death without administration of IFN-γ.

22. The method of claim 19, wherein the risk of death of the individual is at least four-fold less than an expected risk of death without administration of IFN-γ.

23. The method of claim 19, wherein IFN-γ is administered in a dose of about 80 μg/m² to about 90 μg/m².

24. The method of claim 19, wherein IFN-γ is administered in a dose of about 200 μg.

25. The method of claim 23 or 24, wherein IFN-γ is administered three times weekly.

26. The method of claim 25, wherein IFN-γ is administered by subcutaneous administration.

27. A method of treating idiopathic pulmonary fibrosis in an individual, the method comprising the steps of:
(a) ascertaining that the individual has a forced vital capacity (FVC) of at least about 55% of the normal predicted value; and
(b) administering to the individual an effective amount of IFN-γ.

28. The method of claim 27, wherein the probability of survival of the individual is at least about 10% greater than an expected probability of survival without administration of IFN-γ.

29. The method of claim 27, wherein the probability of survival of the individual is at least about 15% greater than an expected probability of survival without administration of IFN-γ.

30. The method of claim 27, wherein the risk of death of the individual is at least about two-fold less than an expected risk of death without administration of IFN-γ.

31. The method of claim 27, wherein IFN-γ is administered in a dose of about 80 μg/m² to about 90 μg/m².

32. The method of claim 27, wherein IFN-γ is administered in a dose of about 200 μg.

33. The method of claim 31 or 32, wherein IFN-γ is administered three times weekly.

34. The method of claim 33, wherein IFN-γ is administered by subcutaneous injection.

35. The method of any of claims 1-34, wherein the individual is a human.
